(19) United States
(12) Patent Application Publication
(10) Pub. No.: US 2021/0297574 A1
(43) Pub. Date: Sep. 23, 2021

(54) CONTROL DEVICE AND MEDICAL OBSERVATION SYSTEM

(71) Applicant: Sony Olympus Medical Solutions Inc., Tokyo (JP)

(72) Inventor: Hiroshi USHIRODA, Tokyo (JP)

(73) Assignee: Sony Olympus Medical Solutions Inc., Tokyo (JP)

(21) Appl. No.: 17/161,678

(22) Filed: Jan. 29, 2021

(30) Foreign Application Priority Data
Mar. 18, 2020 (JP) 2020-047498

(51) Int. Cl. H04N 5/235; H04N 5/225; H04N 7/18; A61B 90/00; A61B 90/30

(52) U.S. Cl. CPC H04N 5/2352; H04N 5/2256; H04N 7/18; A61B 90/25; A61B 90/30; A61B 90/37; A61B 90/361

(57) ABSTRACT

A control device includes a controller configured to: control an image sensor configured to capture an image of a subject; control a light source configured to irradiate the subject with illumination light; and change at least part of an illumination condition of illumination light of an exposure period of a light receiving element of the image sensor and a period other than the exposure period in an image-capturing processing period for acquiring an image signal of an image of one frame to be displayed by a display.

### CROSS-REFERENCE TO RELATED APPLICATION

This application claims priority from Japanese Application No. 2020-047498, filed on Mar. 18, 2020, the contents of which are incorporated by reference herein in its entirety.

### BACKGROUND

The present disclosure relates to a control device and a medical observation system.

In the related art, as a medical observation system for observing a minute part when an operation is to be performed on the minute part of, for example, the brain or the heart of a patient who is an observation object, an optical microscope system provided with a support part having a plurality of arm parts and a microscope part having a magnification optical system and an imaging element provided at a distal end of the support part to magnify the minute part is known (for example, see WO 2016/208485 A). When an operation is to be performed by using this microscope system, an operator (user) such as a doctor moves the microscope part, places the microscope part at a desired position, and performs the operation while observing images captured by the microscope part.

### SUMMARY

Meanwhile, operations involve two observation situations which are image observation by a microscope part and direct-viewing observation of directly observing the observation object. In this case, in the image observation, the observation object is illuminated with illumination light in order to ensure brightness of the image. However, in some cases, bright points are generated in the image because of the illumination light and result in a hard-to-see image, and the operative site may not be easily seen due to reflection, etc. when irradiation with the illumination light for image acquisition is carried out during the direct-view observation.

According to one aspect of the present disclosure, there is provided a control device including a controller configured to: control an image sensor configured to capture an image of a subject; control a light source configured to irradiate the subject with illumination light; and change at least part of an illumination condition of illumination light of an exposure period of a light receiving element of the image sensor and a period other than the exposure period in an image-capturing processing period for acquiring an image signal of an image of one frame to be displayed by a display.

### BRIEF DESCRIPTION OF THE DRAWINGS

FIG. 1 is a diagram illustrating a configuration of a medical observation system according to a first embodiment;

FIG. 2 is a block diagram illustrating a configuration of a control device of the medical observation system according to the first embodiment;

FIG. 3 is a diagram for describing a usage mode of the microscope device of the medical observation system according to the first embodiment;

FIG. 4 is a timing chart describing the image-capturing processing and the illumination processing carried out by the control device of the medical observation system according to the first embodiment;

FIG. 5 is a timing chart describing the image-capturing processing and the illumination processing carried out by the control device of the medical observation system according to the modification example of the first embodiment;

FIG. 6 is a block diagram illustrating a configuration of a control device of a medical observation system according to the second embodiment;

FIG. 7 is a timing chart describing the image-capturing processing and laser-light-emission processing carried out by the control device of the medical observation system according to the second embodiment;

FIG. 8 is a block diagram illustrating a configuration of a control device of a medical observation system according to the third embodiment; and

FIG. 9 is a timing chart describing the image-capturing processing and laser-light-emission processing carried out by the control device of the medical observation system according to the third embodiment.

### DETAILED DESCRIPTION

Hereinafter, with reference to accompanying drawings, embodiments for carrying out the present disclosure (hereinafter, referred to as “embodiments”) will be described. Note that the drawings are merely schematic, and parts having mutually different dimensional relations or ratios may be included among the drawings.

### First Embodiment

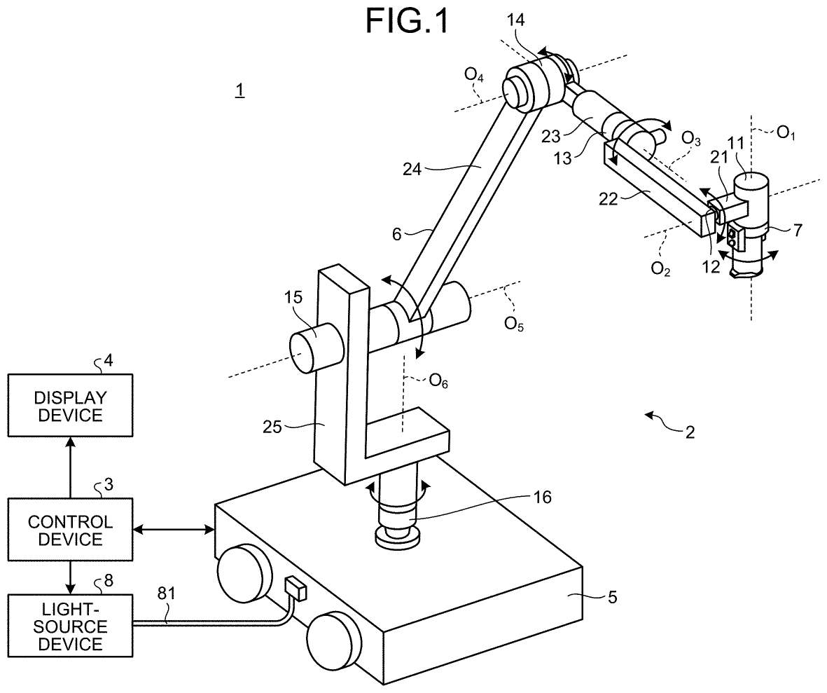

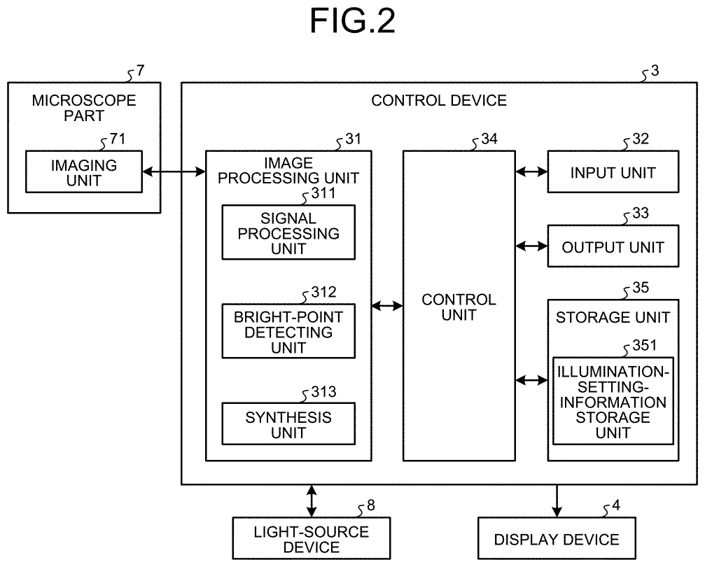

FIG. 1 is a diagram illustrating a configuration of a medical observation system according to a first embodiment. FIG. 2 is a block diagram illustrating a configuration of a control device of the medical observation system according to the first embodiment. A medical observation system 1 is provided with a microscope device 2, which has a function as a microscope which magnifies and captures an image of a fine structure of an observation object; a control device 3, which integrally controls operations of the medical observation system 1; a display device 4, which displays the image captured by the microscope device 2; and a light-source device 8, which supplies illumination light to the microscope device 2.

The microscope device 2 is provided with a base part 5, which may be moved on a floor; a support part 6, which is supported by the base part 5; and a columnar microscope part 7, which is provided at a distal end of the support part 6 and magnifies and captures an image of a minute part of the observation object.

In the microscope device 2, for example, cables including transmission cables including signal lines for carrying out signal transmissions between the control device 3 and the microscope part 7, light guide cables for guiding illumination light from the light-source device 8 to the microscope part 7, etc. are disposed from the base part 5 to the microscope part 7.

The support part 6 has a first joint part 11, a first arm part 21, a second joint part 12, a second arm part 22, a third joint part 13, a third arm part 23, a fourth joint part 14, a fourth arm part 24, a fifth joint part 15, a fifth arm part 25, and a sixth joint part 16.

The support part 6 has four sets, each set including two arm parts and a joint part turnably coupling one of the two arm parts (distal end side) to the other one (proximal end side). Specifically, these four sets are (the first arm part 21, the second joint part 12, and the second arm part 22), (the second arm part 22, the third joint part 13, and the third arm part 23), (the third arm part 23, the fourth joint part 14, and the fourth arm part 24), and (the fourth arm part 24, the fifth joint part 15, and the fifth arm part 25).

The first joint part 11 turnably retains the microscope part 7 in the distal end side and is retained by the first arm part 21 in a state in which it is fixed to a distal end part of the first arm part 21 in the proximal end side. The first joint part 11 has a cylindrical shape and turnably retains the microscope part 7 about a first axis O₁, which is a central axis in a height direction. The first arm part 21 has a shape extending from a lateral surface of the first joint part 11 in a direction orthogonal to the first axis O₁.

The second joint part 12 turnably retains the first arm part 21 in the distal end side and is retained by the second arm part 22 in a state in which it is fixed to a distal end part of the second arm part 22 in the proximal end side. The second joint part 12 has a cylindrical shape and turnably retains the first arm part 21 about a second axis O₂, which is a central axis in a height direction and is an axis orthogonal to the first axis O₁. The second arm part 22 has an approximately L-shape and is coupled to the second joint part 12 by an end of a vertical-line part of the L-shape.

The third joint part 13 turnably retains a horizontal-line part of the L-shape of the second arm part 22 in the distal end side and is retained by the third arm part 23 in a state in which it is fixed to the distal end part of the third arm part 23 in the proximal end side. The third joint part 13 has a cylindrical shape and turnably retains the second arm part 22 about a third axis O₃, which is a central axis in a height direction, is orthogonal to the second axis O₂, and is an axis parallel to the direction in which the second arm part 22 is extending. The third arm part 23 has a distal end side having a cylindrical shape and has a proximal end side in which a hole portion penetrating through the part in the direction orthogonal to the height direction of the cylinder of the distal end side is formed. The third joint part 13 is turnably retained by the fourth joint part 14 via this hole portion.

The fourth joint part 14 turnably retains the third arm part 23 in the distal end side and is retained by the fourth arm part 24 in a state in which it is fixed to the fourth arm part 24 in the proximal end side. The fourth joint part 14 has a cylindrical shape and turnably retains the third arm part 23 about a fourth axis O₄, which is a central axis in a height direction and is an axis orthogonal to the third axis O₃.

The fifth joint part 15 turnably retains the fourth arm part 24 in the distal end side and is attached to the fifth arm part 25 in the proximal end side. The fifth joint part 15 has a cylindrical shape and turnably retains the fourth arm part 24 about a fifth axis O₅, which is a central axis in a height direction and is an axis parallel to the fourth axis O₄. The fifth arm part 25 includes a part forming an L-shape and a rod-like part extending downward from a horizontal-line part of the L-shape. The fifth joint part 15 is attached to an end of a vertical-line part of the L-shape of the fifth arm part 25 in the proximal end side.

The sixth joint part 16 turnably retains the fifth arm part 25 in the distal end side and is fixedly attached to an upper surface of the base part 5 in the proximal end side. The sixth joint part 16 has a cylindrical shape and turnably retains the fifth arm part 25 about a sixth axis O₆, which is a central axis in a height direction and is an axis orthogonal to the fifth axis O₅. A base end part of the rod-like part of the fifth arm part 25 is attached to the distal end side of the sixth joint part 16.

The support part 6 having the above described configuration realizes six degrees of freedom in total including three degrees of translational freedom and three degrees of rotational freedom of the microscope part 7.

The first joint part 11 to the sixth joint part 16 have electromagnetic brakes which forbid turning of the microscope part 7 and the first arm part 21 to the fifth arm part 25, respectively. The electromagnetic brakes are released in a state, in which an arm operation switch (described later) provided on the microscope part 7 is pressed down, and allow turning of the microscope part 7 and the first arm part 21 to the fifth arm part 25. Note that air brakes may be applied instead of the electromagnetic brakes.

Each of the joint parts may be equipped with an encoder and an actuator other than the above described electromagnetic brake. If the encoder is provided, for example, at the first joint part 11, the encoder detects a rotation angle about the first axis O₁. The actuator includes, for example, an electric motor such as a servo motor, is driven by control from the control device 3, and causes rotation at the joint part by a predetermined angle. The rotation angle at the joint part is set by the control device 3 based on the rotation angle about each of the rotation axes (the first axis O₁ to the sixth axis O₆), for example, as a value necessary for moving the microscope part 7. In this manner, the joint part provided with an active driving system such as an actuator constitutes a rotation shaft which actively rotates when driving of the actuator is controlled.

The microscope part 7 has, in a casing having a cylindrical shape, an imaging unit 71 which magnifies and captures an image of the observation object. Other than that, the microscope part 7 is provided with the arm operation switch, which receives operation inputs to release the electromagnetic brakes of the first joint part 11 to the sixth joint part 16 and allow turning of the joint parts, and a cross-shaped lever, which may change magnification power of the imaging unit and a focal length from the observation object. While the arm operation switch is pressed down by the user, the electromagnetic brakes of the first joint part 11 to the sixth joint part 16 are released.

The imaging unit 71 captures an image of a subject under control of a camera-head control unit 94. The imaging unit 71 is constituted by housing a plurality of lenses and an imaging element in the casing. The imaging element has a global shutter function of reading electric charges of light receiving elements serving as reading targets at one time, receives the light of a subject image formed by the lenses, and converts the light to electric signals (image capture signals). The imaging unit 71 forms an observation optical system, which forms the subject image passed through the lenses on an imaging surface of the imaging element. The imaging element includes a charge coupled device (CCD) image sensor or a complementary metal oxide semiconductor (CMOS) image sensor.

The light-source device 8 controls emission of light under control of the control device 3. The light-source device 8 is connected to the microscope device 2 via a light-source cable 81. An optical fiber is inserted in the light-source cable 81.

The control device 3 receives the image capture signal output by the microscope device 2 and generates image data to be displayed by subjecting the image capture signal to predetermined signal processing. Note that the control device 3 may be installed in the base part 5 and integrated with the microscope device 2.

The control device 3 is provided with an image processing unit 31, an input unit 32, an output unit 33, a control unit 34, and a storage unit 35. Note that the control device 3 may be provided with, for example, a power source unit (illustration omitted), which generates a power-source voltage for driving the microscope device 2 and the control device 3, supplies the power-supply voltage to each unit of the control device 3, and supplies the power-source voltage to the microscope device 2 via the transmission cable.

The image processing unit 31 subjects the image capture signal, which has been output from the microscope part 7, to processing to generate the image to be displayed. The image processing unit 31 has a signal processing unit 311, a bright-point detecting unit 312, and a synthesis unit 313.

The signal processing unit 311 carries out noise removal and signal processing such as A/D conversion, detection processing, interpolation processing, and/or color correction processing in accordance with needs. The signal processing unit 311 generates image signals based on the image capture signals which have undergone signal processing. In the present embodiment, the image signals of one display frame is generated by synthesizing the image signals of two continuous captured image frames captured by the imaging unit 71. Therefore, the image signals generated by the signal processing unit 311 may be considered as those of a frame having approximately a half information quantity as a display frame.

The bright-point detecting unit 312 detects pixel values, which are expressed as bright points, from the image signals generated by the signal processing unit 311 and detects them as bright points. The bright-point detecting unit 312 detects the bright points by comparing a threshold value, which is a threshold value set in advance and set in accordance with pixel values which may be expressed as bright points in images, with each of pixel values of the image signals.

The synthesis unit 313 synthesizes the image signals of two captured image frames, which are temporally continuous, to generate the image signals of one display frame. The synthesis unit 313, for example, replaces the pixel values of the positions of the bright points detected by the bright-point detecting unit 312 in one of the frames with the pixel values of the corresponding position in the other frame. The image signals generated by the synthesis unit 313 are output to the display device 4 and displayed by the display device 4.

Also, the image processing unit 31 may have an AF processing unit, which outputs predetermined AF evaluation values of each of the frames based on the image capture signals of the input frames, and an AF calculation unit, which carries out AF calculation processing so as to select, for example, a frame most appropriate as a focal position or a focus lens position from the AF evaluation values of the frames from the AF processing unit.

The input unit 32 is realized by using a user interface(s) such as a keyboard, a mouse, and/or a touch panel and receives input of various information.

The output unit 33 is realized by using, for example, a speaker, a printer, and/or a display and outputs various information.

The control unit 34 carries out, for example, drive control of constituent units including the microscope device 2, the control device 3, and the light-source device 8 and input/output control of information with respect to the constituent units. The control unit 34 generates control signals by referencing communication information data (for example, communication format information, etc.) recorded in the storage unit 35 and transmits the generated control signals to the microscope device 2.

Note that the control unit 34 generates synchronization signals and clock signals of the microscope part 7 and the control device 3. The synchronization signals (for example, synchronization signals for instructing image capture timing) and the clock signals (for example, clock signals for serial communication) for the microscope part 7 are transmitted to the microscope part 7 by an unillustrated line, and the microscope part 7 is driven based on these synchronization signals and the clock signals.

The storage unit 35 is realized by using a semiconductor memory such as a flash memory or a dynamic random access memory (DRAM), and communication information data (for example, communication format information), etc. is recorded therein. Note that various programs, etc. executed by the control unit 34 may be recorded in the storage unit 35.

Also, the storage unit 35 has an illumination-setting-information storage unit 351. The illumination-setting-information storage unit 351 stores setting information of a light intensity of illumination light of the captured image frame in an exposure period and a light intensity of illumination light in a period in which an operator directly views an operative site other than an exposure period.

The image processing unit 31 and the control unit 34 described above are realized by using a general-purpose processor(s) such as a central processing unit (CPU) having an internal memory (illustration omitted) in which a program(s) is recorded or a dedicated processor(s) such as a various calculation circuit which executes a particular function(s) like an application specific integrated circuit (ASIC). Also, the units may be constituted by using a field programmable gate array (FPGA: illustration omitted), which is a type of a programmable integrated circuit. Note that, if the unit is constituted by a FPGA, a memory for storing configuration data may be provided, and the FPGA which is a programmable integrated circuit may be configured by the configuration data read from the memory.

The display device 4 receives the image data, which has been generated by the control device 3, from the control device 3 and displays an image corresponding to the image data. The display device 4 like this is provided with a display panel including liquid crystals or organic electro luminescence (EL). Note that, other than the display device 4, an output device which outputs information by using a speaker, a printer, or the like may be provided.

Outlines of an operation carried out by using the medical observation system 1 having the above described configuration will be described. If an operator who is the user is to perform an operation on the head of a patient who is an observation object, the operator holds the microscope part 7 and moves the microscope part 7 to a desired position in a state in which the arm operation switch of the microscope part 7 is pressed down while the operator sees the image displayed by the display device 4, determines an imaging view field of the microscope part 7, and then detaches his/her finger from the arm operation switch. As a result, the electromagnetic brakes work at the first joint part 11 to the sixth joint part 16, and the imaging view field of the microscope part 7 is fixed. Then, the operator carries out, for example, adjustment of the magnification power and the focal length to the observation object.

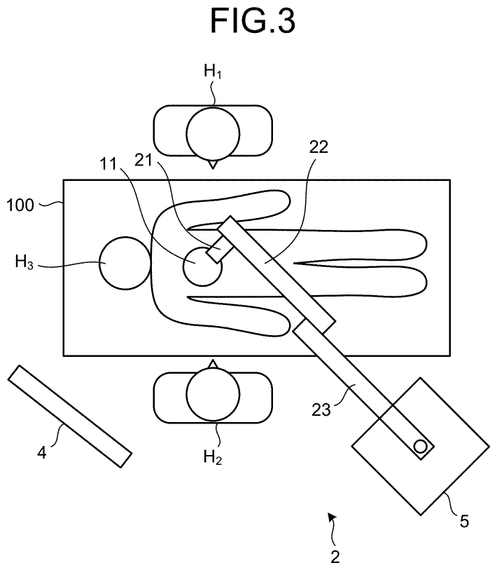

FIG. 3 is a diagram for describing a usage mode of the microscope device of the medical observation system. Note that FIG. 3 illustrates a state in which a situation in an operation is viewed from immediately above. An operator H₁ performs an operation while observing video of an operative site shown by the display device 4. The operator H₁ performs an operation on a patient H₃ lying on an operating table 100 by using the microscope device 2. Other than the operator H₁ who performs the operation, FIG. 3 also illustrates an assistant H₂ who assists the operation. Note that the present first embodiment illustrates an example in which the display device 4 is installed so as to be positioned approximately in front of the operator H₁ when he/she performs the operation. In the operation, the operator H₁ checks the image displayed by the display device 4 and/or directly observes the operative site of the patient H₃ to perform the surgery.

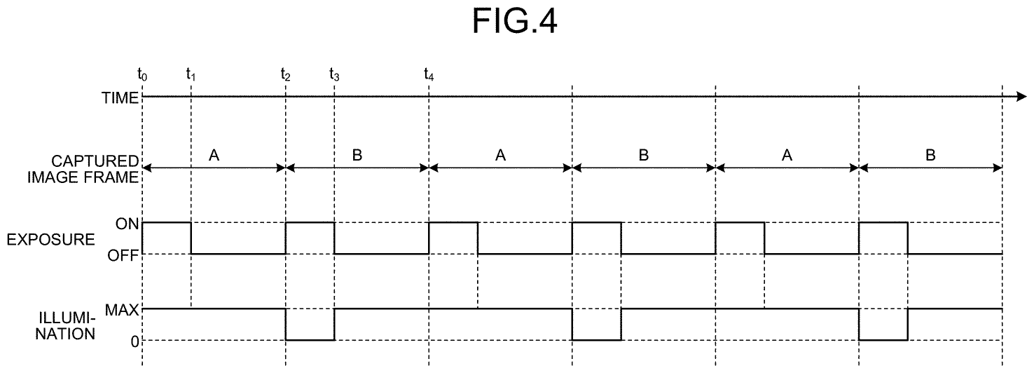

Next, image-capturing processing and illumination processing of the present first embodiment will be described with reference to FIG. 4. FIG. 4 is a timing chart describing the image-capturing processing and the illumination processing carried out by the control device of the medical observation system according to the first embodiment. Hereinafter, the description will be given on an assumption that the units operate under control of the control unit 34. Note that, in the example illustrated in FIG. 4, an image signal for display is generated by synthesizing a captured image frame A and a captured image frame B subsequent to that. In the present first embodiment, the captured image frame A and the captured image frame B correspond to an image-capturing processing period for generating the image signal of one frame for display. Note that, FIG. 4 will be described on an assumption that time equal to or more than the time for exposure processing and reading processing is set for each of the periods of the captured image frames A and B of the captured image frames. Also, the time chart illustrated in FIG. 4 will be described as an example of the processing in an operation room as illustrated in FIG. 3.

At time t₀, exposure of the captured image frame A is started (exposure: ON). At the same time, the light-source device 8 emits illumination light having a light intensity for exposure. In the example of FIG. 4, the light intensity for exposure is set to a maximum value (MAX). As a result, an operative site is illuminated with the light intensity for exposure, and exposure processing is carried out at the imaging element.

Note that the light intensity for exposure may be appropriately set based on the brightness of the image acquired in previous image capturing under control of the control unit 34.

When the exposure processing of the captured image frame A is finished (exposure: OFF) at time t₁, read processing is started. At the same time, the light-source device 8 maintains emission of illumination light with the light intensity for direct viewing. In the example illustrated in FIG. 4, the light intensity for exposure (image acquisition) and the light intensity for direct viewing are the same light intensity (maximum value (MAX)).

The emission of the illumination light is continued to time t₂ at which the set period of the captured image frame A is finished. For example, the operator directly observes the operative site from the time t₁ to the time t₂ (hereinafter, also referred to as non-exposure period) corresponding to the period other than the exposure processing.

Then, at the time t₂, exposure of the captured image frame B is started. The captured image frame B is a frame for acquiring an image for removing bright points in the image which may be generated in the captured image frame A. Therefore, in the captured image frame B, the emission of the illumination light by the light-source device 8 is turned off, and the operative site is illuminated, for example, only with an illumination device of the operating room. The image captured in the captured image frame B is an image having lower brightness than the image captured in the captured image frame A.

When the exposure processing in the captured image frame B is finished at time t₃, read processing is started. At the same time, the light-source device 8 emits illumination light having a light intensity for direct viewing. The light intensity for direct viewing in this case is the same as the light intensity of the captured image frame A for direct viewing (maximum value (MAX)).

The emission of the illumination light is continued to time t₄ at which the set period of the captured image frame B finishes.

In this manner, in the first embodiment, the light intensity of the illumination light is changed within the continued periods from the captured image frame A to the captured image frame B. Also, in the first embodiment, in order to suppress flickering upon switching of the light intensities in the captured image frame B, it is preferred to execute the light intensity switching operation of illumination light at 100 fps or higher.

After the time t₄, the image-capturing processing and the illumination processing of the captured image frame A and the captured image frame B is repeated as well as the time t₀ to t₄ described above.

Herein, when the image-capturing processing of the captured image frame A is finished, the signal processing unit 311 generates an image signal based on the image signal of the captured image frame A, and the bright-point detecting unit 312 carries out detection processing of bright points of the image based on the generated image signal.

Also, when the image-capturing processing of the captured image frame B is finished, the synthesis unit 313 synthesizes the image signal of the captured image frame A with the image signal of the captured image frame B to generate an image signal for display. In this case, the synthesis unit 313 generates the image signal in which the pixel values of the positions of the bright points detected in the image of the captured image frame A are replaced by the pixel values of the image of the captured image frame B.

In the first embodiment described above, the image signals of the captured image frames having different light intensities in exposure are synthesized, the image signal for display in which the bright points generated in one of the images are replaced by the pixel values of the other image is generated, and the operative site is illuminated with the light having the light intensity set for direct viewing other than during the exposure processing. According to the present first embodiment, the image in which the bright points of the images have been eliminated is generated, and the illumination for direct viewing is also ensured. Therefore, appropriate illumination may be implemented for both of the observations including the observation by the captured image and the observation by direct viewing.

Note that, in the first embodiment, the synthesis unit 313 may be configured to generate the image for display (HDR image) by subjecting the image of the captured image frame A and the image of the captured image frame B to high dynamic range synthesis. In this case, the image processing unit 31 may be configured not to have the bright-point detecting unit 312 so that the synthesis unit 313 generates the HDR image by using the image signal generated by the signal processing unit 311, or the image processing unit 31 may be configured to have the bright-point detecting unit 312 so that the synthesis unit 313 enlarges the components of the captured image frame B at the bright point positions detected by the bright-point detecting unit 312 to synthesize the images.

Also, in the first embodiment, the illumination directions of the illumination light in the exposure period and the non-exposure period of the captured image frames may be configured to be mutually different directions. In this case, the light intensities of the illumination light in the frames may be the same or mutually different light intensities. Note that the “illumination direction” referred to herein corresponds to the direction in which the optical axis of light extends.

Also, in the first embodiment, the illumination light may be polarized light, and the directions of the polarized light of the illumination light may be switched between the exposure period and the non-exposure period of the captured image frames. In this case, for example, the imaging element is provided with an image-capturing polarization element, the light-source device is provided with an illumination polarization element, and the polarization state of at least one of the image-capturing polarization element and the illumination polarization element is controlled to a different state depending on the exposure period or non-exposure period. By virtue of this, the illumination having mutually different polarization directions are implemented in the captured image frames.

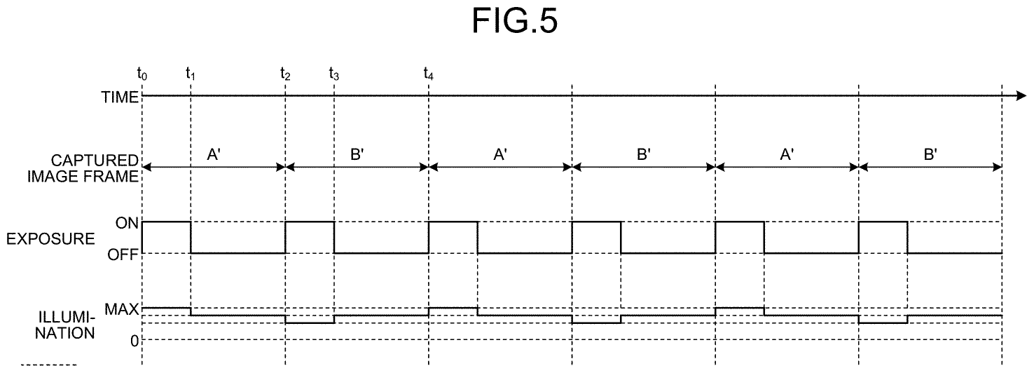

Modification Example of First Embodiment Next, a modification example of the first embodiment will be described with reference to FIG. 5. FIG. 5 is a timing chart describing the image-capturing processing and the illumination processing carried out by the control device of the medical observation system according to the modification example of the first embodiment. Since the configuration of the medical observation system according to a second present embodiment is the same configuration as the medical observation system 1 of the above described first embodiment, description thereof will be omitted. Hereinafter, image-capturing processing and illumination processing different from the first embodiment will be described. In the modification example, an example in which the light intensity of the illumination light for direct viewing is smaller than the light intensity for exposure processing of the captured image frame A will be described.

Then, the exposure processing and the illumination processing of the present modification example will be described with reference to FIG. 5. Note that, in the example illustrated in FIG. 5, an image signal for display is generated by synthesizing a captured image frame A′ and a captured image frame B′ subsequent to that. In the present modification example, the captured image frame A′ and the captured image frame B′ correspond to an image-capturing processing period for generating the image signal of one frame for display. Note that, FIG. 5 will be described on an assumption that time equal to or more than the time for exposure processing and reading processing is set for each of the periods of the captured image frames A′ and B′. Also, the time chart illustrated in FIG. 5 will be described as an example of the processing in an operation room as illustrated in FIG. 3.

At time t₀, exposure of the captured image frame A′ is started. At the same time, the light-source device 8 emits illumination light having a light intensity for exposure (the maximum value (MAX) in the example of FIG. 5). As a result, an operative site is illuminated with the light intensity for exposure, and exposure processing is carried out at the imaging element.

When the exposure processing in the captured image frame A′ is finished at time t₁, read processing is started. At the same time, the light-source device 8 reduces the light intensity of the illumination light. Since the light intensity for direct viewing is reduced more than the light intensity for imaging, the operator may be prevented from feeling blinding because of the operative site illuminated with an excessive light intensity.

The emission of the illumination light is continued to time t₂ at which the set period of the captured image frame A′ is finished.

Then, exposure of the captured image frame B′ is started at the time t₂. The captured image frame B′ is a frame for acquiring an image for removing bright points in the image which may be generated in the captured image frame A′. Therefore, in the captured image frame B′, the emission of the illumination light by the light-source device 8 is set to the light intensity which is lower than the light intensity for direct viewing. The image captured in the captured image frame B′ is an image having lower brightness than the image captured in the captured image frame A′.

When the exposure processing in the captured image frame B′ is finished at time t₃, read processing is started. At the same time, the light-source device 8 emits illumination light having a light intensity for direct viewing. The light intensity for direct viewing in this case is the same as the light intensity for direct viewing of the captured image frame A′.

The emission of the illumination light is continued until time t₄ at which the set period of the captured image frame B′ is finished.

After the time t₄, the image-capturing processing and the illumination processing of the captured image frame A′ and the captured image frame B′ is repeated as well as the time t₀ to t₄ described above.

Herein, when the image-capturing processing of the captured image frame A′ is finished, the signal processing unit 311 generates an image signal based on the image signal of the captured image frame A′, and the bright-point detecting unit 312 carries out detection processing of bright points of the image based on the generated image signal.

Also, when the image-capturing processing of the captured image frame B′ is finished, the synthesis unit 313 synthesizes the image signal of the captured image frame A′ with the image signal of the captured image frame B′ to generate an image signal for display in a manner similar to that of the first embodiment.

In the modification example described above, the image signals of the captured image frames having different light intensities in exposure are synthesized, the image signal for display in which the bright points generated in one of the images are replaced by the pixel values of the other image is generated, and the operative site is illuminated with the light having the light intensity set for direct viewing, the light intensity smaller than the light intensity of the captured image frame A′ and larger than the light intensity of the captured image frame B′ other than during the exposure processing. According to the present modification example, the image in which the bright points of the images have been eliminated is generated, and the illumination for direct viewing is also set to an appropriate light intensity. Therefore, appropriate illumination may be implemented for both of the observations including the observation by the captured image and the observation by direct viewing.

Note that, in the above described modification example, the illumination by the light-source device 8 may be configured to be stopped in the period other than the exposure processing. If the illumination by the light-source device 8 is stopped, the operative site is illuminated with the light from a light source provided in the operating room or outside light. In this manner, the light intensity of the light-source device 8 in the period other than the exposure processing may be appropriately set so that the light intensity is appropriate for direct viewing.

### Second Embodiment

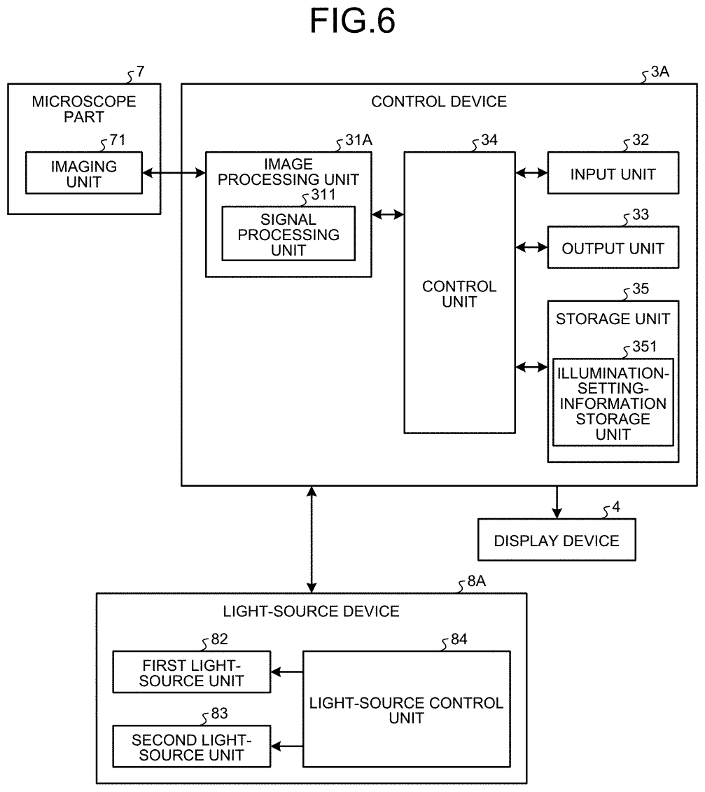

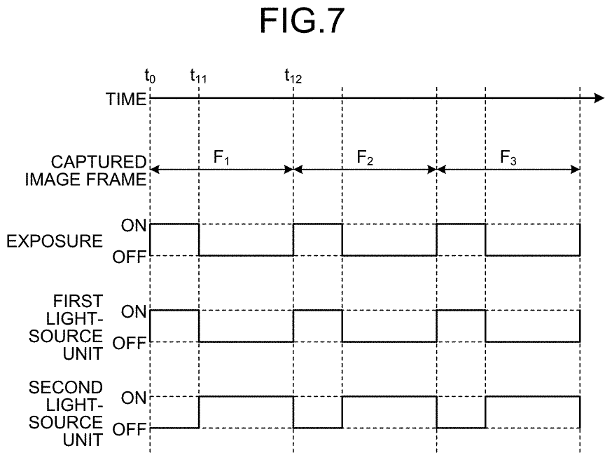

Next, a second embodiment will be described with reference to FIG. 6 and FIG. 7. FIG. 6 is a block diagram illustrating a configuration of a control device of a medical observation system according to the second embodiment. The configuration of the medical observation system according to the present second embodiment is provided with a control device 3A and a light-source device 8A in place of the control device 3 and the light-source device 8 of the above described medical observation system 1. Hereinafter, the components and processing different from the first embodiment will be described. Note that the components which are the same as those of the above described first embodiment are denoted with the same reference signs.

The control device 3A is provided with an image processing unit 31A, an input unit 32, an output unit 33, a control unit 34, and a storage unit 35. The image processing unit 31A is configured to have only the above described signal processing unit 311.

The light-source device 8A is provided with a first light-source unit 82, a second light-source unit 83, and a light-source control unit 84.

The first light-source unit 82 supplies excitation light, which excites an observation object, to the microscope device 2 under control of the light-source control unit 84. This excitation light is the light in a wavelength range different from a wavelength band of white light (visible light) including the light of the wavelength band of a visible range. Specifically, the excitation light is the light in a wavelength range which is part of the wavelength ranges of visible light (for example, part of the wavelength band of green, part of the wavelength band of blue, or a wavelength band which is a combination thereof) or the light of a wavelength range (for example, infrared) outside of the wavelength range of visible light and is used in special light observation.

The special light observation includes:

narrow band imaging (NBI) in which the state of blood vessels of a superficial mucosa membrane and layers deeper than that is observed by irradiation with illumination light having a narrow band having a central wavelength at wavelengths 415 nm and 540 nm and utilizing the difference of absorption of the light of each wavelength with respect to hemoglobin;

IRI in which a medical agent called Indocyanine green (ICG) having an absorption peak in near infrared light in a vicinity of a wavelength of 805 nm in blood is subjected to intravenous injection as a contrast agent, irradiation with excitation light having a central wavelength in a vicinity of 805 nm is carried out, and fluorescence from ICG is observed to diagnose presence/absence of blood flows;

AFI in which a fluorescent agent is injected into a subject in advance, a fluorescent image emitted from the subject when the subject is irradiated with excitation light is observed, and the presence/absence and shape of the fluorescent image is observed to diagnose a tumor part;

PDD in which an image in which cancer cells and normal cells may be easily distinguished from each other by utilizing a property that a solution of aminolevulinic acid (5-ALA) taken by a patient is metabolized into a blood raw material (heme) in normal tissues of the body, but is not metabolized in cancer cells, but accumulated as a substance called PpIX which is an intermediate product thereof, and emits fluorescent light of red (peak wavelength 630 nm) when this PpIX is irradiated with blue light (central wavelength 410 nm); and

infrared light observation in which irradiation with excitation light having an excitation wavelength of 740 nm is carried out, and fluorescence having a wavelength of 830 nm is detected.

The second light-source unit 83 supplies the light of a wavelength band of white light (visible light) including the wavelength band of a visible region to the microscope device 2 under control of the light-source control unit 84.

In the microscope device 2, for example, a window for emitting the light of the first light-source unit 82 and a window for emitting the light of the second light-source unit 83 are provided at mutually different positions. In this case, the illumination directions of the light emitted by the light-source units are mutually different.

The light-source control unit 84 controls light emission of the first light-source unit 82 and the second light-source unit 83 under control of the control device 3A. The light-source control unit 84 includes a memory and a processor having hardware such as CPU, ASIC, FPGA, etc.

Next, exposure processing and illumination processing of the present second embodiment will be described with reference to FIG. 7. FIG. 7 is a timing chart describing the image-capturing processing and laser-light-emission processing carried out by the control device of the medical observation system according to the second embodiment. Note that, in the example illustrated in FIG. 7, images are generated in respective captured image frames. Specifically, the signal processing unit 311 generates image signals for display by using image signals generated in sequentially-captured captured image frames F₁, F₂, F₃, and so on. In the present second embodiment, each of the captured image frames (captured image frames F₁, F₂, F₃, and so on) corresponds to an image-capturing processing period for generating the image signal of one frame for display.

At time t₀, exposure of the captured image frame F₁ is started (exposure: ON). In the present second embodiment, in exposure processing, excitation light is emitted from the first light-source unit 82. Therefore, a fluorescent substance introduced into an observation object is excited, and fluorescence is emitted. The imaging unit 71 captures this fluorescent image.

When the exposure processing of the captured image frame F₁ is finished (exposure: OFF) at time t₁₁, read processing is started. At the same time, the light-source device 8A maintains emission of illumination light with the light intensity for direct viewing. In the second embodiment, the second light-source unit 83 emits white light.

The emission of the white light is continued to time t₁₂ at which the set period of the captured image frame F₁ finishes. For example, the operator directly observes the operative site from the time t₁₁ to the time t₁₂ corresponding to the period other than the exposure processing. In this manner, in the second embodiment, the type of the illumination light is changed among the captured image frames.

In the second embodiment, in order to suppress flickering upon switching of the illumination light in the captured image frame, it is preferred to execute the switching operation of illumination light at 100 fps or higher as well as the first embodiment.

Then, similar processing is repeated, image signals of the captured image frames such as the captured image frames F₂, F₃, and so on are generated, and irradiation with white light is carried out other than the exposure period. Note that the type of the excitation light with which irradiation is carried out may be changed, and the observation object may be changed. Also, the light intensity of white light may be appropriately adjusted via, for example, the input unit 32.

In the second embodiment described above, the observation object is configured to be irradiated with the excitation light, which excites a fluorescent substance, in exposure processing, and the observation object is configured to be irradiated with white light other than the exposure processing period. Therefore, a fluorescent image in which the fluorescent substance is excited is acquired, and the operative site is illuminated by irradiation of the white light. Therefore, the fluorescence which may not be easily directly observed may be observed by the image, and the operative site may be observed in a state in which it is illuminated with the white light.

Note that, in the above described second embodiment, whether synchronization control of the light-source units is valid/invalid may be switched. If the synchronization control is invalid, each of the light-source units is individually controlled in a similar manner. Also, if the synchronization control is valid, each of the light-source units increases a gain compared with the invalid case in order to increase sensitivity and shorten exposure time. By virtue of this, the time of the exposure period is shortened, and the time other than the exposure period is extended, in other words, the illumination time for direct viewing observation is extended.

Also, for example, in a case in which IRI is configured to be performed in the above described second embodiment, if a filter which blocks the light of a wavelength band equal to or less than the wavelength band of the excitation light may be inserted/removed to/from a light receiving surface of the imaging element or if the imaging unit has an imaging element which receives fluorescence and an imaging element which receives white light, the illumination light for direct viewing may be emitted at the same time when irradiation with the excitation light is carried out. By virtue of this, in one frame period, irradiation with the illumination light for direct viewing is carried out in the whole period, and irradiation with the excitation light is carried out only at the timing for exposure. Therefore, unnecessary energy irradiation of the operative site with the excitation light may be suppressed.

### Third Embodiment

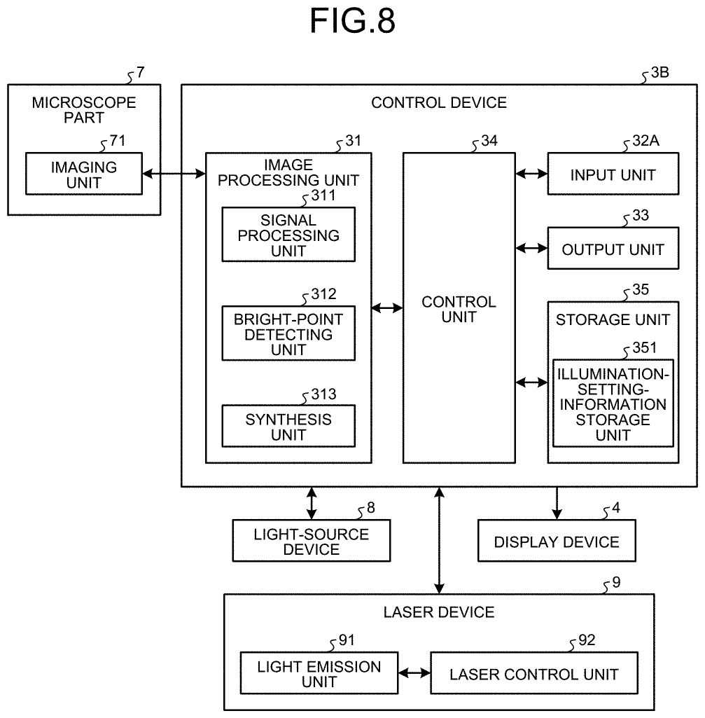

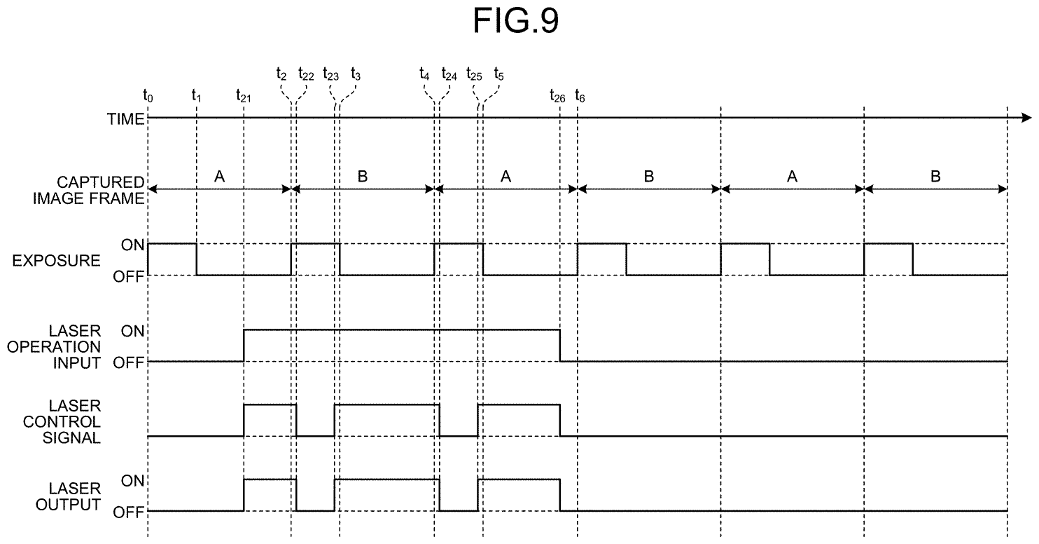

Next, a third embodiment will be described with reference to FIG. 8 and FIG. 9. FIG. 8 is a block diagram illustrating a configuration of a control device of a medical observation system according to the third embodiment. The configuration of the medical observation system according to the present third embodiment is provided with a control device 3B in place of the control device 3 of the above described medical observation system 1 and is further provided with a laser device 9. Hereinafter, the components and processing different from the first embodiment will be described. Note that the components which are the same as those of the above described first embodiment are denoted with the same reference signs.

The control device 3B is provided with an image processing unit 31, an input unit 32A, an output unit 33, a control unit 34, and a storage unit 35. The input unit 32A is realized by using a user interface(s) such as a keyboard, a mouse, and/or a touch panel and an operation button for operating laser emission of the laser device 9 and receives input/output of various information.

The laser device 9 is provided with a light emission unit 91 and a laser control unit 92.

The light emission unit 91 emits laser light under control of the laser control unit 92.

The laser control unit 92 carries out emission control of laser light by driving the light emission unit 91 under control of the control device 3B.

Next, exposure processing and illumination processing of the present third embodiment will be described with reference to FIG. 9. FIG. 9 is a timing chart describing the image-capturing processing and laser-light-emission processing carried out by the control device of the medical observation system according to the third embodiment. Note that, in the example illustrated in FIG. 9, an image signal for display is generated by synthesizing a captured image frame A and a captured image frame B subsequent to that as well as the first embodiment. The illumination processing by the light-source device 8 may implement illumination by light having a uniform light intensity, or the light intensity may be changed between the period in which the exposure processing is carried out and the period other than the exposure processing like the first embodiment and the modification example. Note that, FIG. 9 will be described on an assumption that time equal to or more than the time for exposure processing and reading processing is set for each of the periods of the captured image frames A and B of the captured image frames. Also, the time chart illustrated in FIG. 9 will be described as an example of the processing in an operation room as illustrated in FIG. 3.

At time t₀, exposure of the captured image frame A is started. Then, when the exposure processing in the captured image frame A is finished at time t₁, read processing is started. Thereafter, at time t₂ to t₄, exposure processing corresponding to the captured image frames A and B is carried out as well as the first embodiment. Then, the exposure processing of the captured image frame A is carried out from time t₄ to time t₅, and processing of the captured image frame B is carried out from time t₆. Then, processing of the captured image frames A and B is repeated.

Herein, if an emission order of the laser device 9 is input via the input unit 32A, the control unit 34 carries out emission control of laser light corresponding to the period of the exposure processing. Specifically, for example even if the operation button is pressed down and emission of laser light is ordered, emission of the laser light is stopped during the exposure processing except during an exposure start period and an exposure end period.

For example, in the example illustrated in FIG. 7, a laser operation order is input at time t₂₁ which is after the time t₁ and before the time t₂, and the laser operation order is continuously input until time t₂₆ between the time t₅ and the time t₆.

In this process, the laser control unit 92 causes the light emission unit 91 to emit laser light until time t₂₂ which is after the time t₂, at which the exposure processing of the captured image frame B is started, by predetermined time and then stops the emission. Then, the laser control unit 92 causes the light emission unit 91 to emit laser light again from time t₂₃ which is before the time t₃, at which the exposure processing of the captured image frame B is finished, by predetermined time. Furthermore, the laser control unit 92 causes the light emission unit 91 to emit laser light until time t₂₄ which is after the time t₄, at which the exposure processing of the captured image frame A is started, by predetermined time and then stops the emission. Then, the laser control unit 92 causes the light emission unit 91 to emit laser light again from time t₂₅ which is before the time t₅, at which the exposure processing of the captured image frame A is finished, by predetermined time and continues the emission of the laser light until time t₂₆.

Note that the “predetermined time” for emitting the laser light during the exposure processing may be set to be the same time or may be set to be different time in the exposure processing start period and the exposure processing end period.

In the third embodiment described above, if the laser operation order is input during the exposure processing, the laser light is configured to be emitted only during a partial period of the exposure processing period. Therefore, an image in which the light receiving amount of laser light, which has a larger light intensity (intensity) than the illumination light for illuminating the subject, is reduced is generated. By virtue of this, laser light may be expressed by the image, and the brightness thereof may be suppressed to a degree at which the brightness does not cause whiteout.

### OTHER EMBODIMENTS

Variations may be formed by appropriately combining plural constituent elements disclosed in the medical observation system according to the embodiments described above. For example, some of the constituent elements may be removed from all the constituent elements described in the medical observation system according to the embodiment described above. Furthermore, the constituent elements described in the medical observation systems according to the first to third embodiments described above may be appropriately combined.

Also, in the medical observation system according to the embodiment, the example in which the image signals corresponding to two captured image frames are synthesized to generate the image of one display frame has been described. However, an image of one display frame may be generated by the image signal of one captured image frame. In that case, for example, the image-capturing processing of the captured image frame A′ is repeated, and an image of one display frame is generated based on the image signal acquired as the captured image frame A′.

Also, in the medical observation system according to the embodiment, the above described “unit” may be replaced by “means”, “circuit”, or the like. For example, the control unit may be replaced by a control means or a control circuit.

Also, a program executed by the medical observation system according to the embodiment is provided by being recorded in a computer-readable recording medium such as a CD-ROM, a flexible disk (FD), a CD-R, a digital versatile disk (DVD), a USB medium, or a flash memory as file data having an installable format or an executable format.

Also, the program executed by the medical observation system according to the embodiment may be stored in a computer connected to a network such as the Internet so that the program is provided by being downloaded via the network.

Hereinabove, some of the embodiments of the present application have been described in detail based on drawings. However, these are examples, and the present disclosure may be implemented by various modifications and modified other modes based on the knowledge of those skilled in the art in addition to the modes described in the disclosure.

Note that the present technique may also employ the following configurations.

(1)

A control device including

a controller configured to:
control an image sensor configured to capture an image of a subject;
control a light source configured to irradiate the subject with illumination light; and
change at least part of an illumination condition of illumination light of an exposure period of a light receiving element of the image sensor and a period other than the exposure period in an image-capturing processing period for acquiring an image signal of an image of one frame to be displayed by a display.
   (2)

The control device according to (1), wherein the controller is configured to control the light source to emit light such that a light intensity of the illumination light in the exposure period and the light intensity in the period other than the exposure period are mutually different light intensities.

(3)

The control device according to (1), wherein the controller is configured to

control the light source to emit white light in the period other than the exposure period, and

control the light source to emit light having a wavelength band different from at least the white light in the exposure period.

(4)

The control device according to (3), wherein the light emitted by the light source in the exposure period is light of a wavelength range serving as part of a wavelength range of visible light.

(5)

The control device according to (3), wherein the light emitted by the light source in the exposure period is light in a wavelength range outside of a wavelength range of visible light.

(6)

The control device according to (1), further including an image processor configured to:

synthesize the image signals of the two temporally-continuous captured image frames to generate a display image for the display device;

detect a bright point position based on the image signal of one of the captured image frames; and

replace a pixel value of the detected bright point position with a pixel value of a position corresponding to the image signal of another captured image frame to synthesize the image signals of the two captured image frames.

(7)

The control device according to (6), wherein the controller is configured to:

control the light source to emit the illumination light in exposure processing of one of the captured image frames; and,

control the light source, in exposure processing of the other captured image frame, to emit the illumination light having a lower light intensity than the illumination light of the exposure processing of the one of the captured image frames.

(8)

The control device according to (7), wherein emission of the illumination light is stopped in the exposure processing of the other captured image frame.

(9)

The control device according to any one of (1) to (8), wherein the image sensor has a global shutter function configured to read an electric charge of the light receiving element serving as a reading target at one time.

(10)

A medical observation system including:

an imaging device including an image sensor configured to capture an image of a subject;

a support configured to support the imaging device;

a light source configured to irradiate the subject with illumination light;

a control device configured to control the imaging device and the light source; and

a display configured to display the image captured by the imaging device, wherein

the control device is configured to change at least part of an illumination condition of the illumination light of an exposure period of a light receiving element of the image sensor and a period other than the exposure period in an image-capturing processing period for acquiring an image signal of a one frame image displayed by the display.

As described above, the control device and the medical observation system according to the present disclosure are effective to implement the illumination appropriate for both of the observations, which are the observation by the captured image and the observation by direct viewing.

According to the present disclosure, effects that appropriate illumination may be implemented for both of the observations, which are the observation by the captured images and the observation by direct viewing are exerted.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A control device comprising
a controller configured to:
control an image sensor configured to capture an image of a subject;
control a light source configured to irradiate the subject with illumination light; and
change at least part of an illumination condition of illumination light of an exposure period of a light receiving element of the image sensor and a period other than the exposure period in an image-capturing processing period for acquiring an image signal of an image of one frame to be displayed by a display.

2. The control device according to claim 1, wherein the controller is configured to control the light source to emit light such that a light intensity of the illumination light in the exposure period and the light intensity in the period other than the exposure period are mutually different light intensities.

3. The control device according to claim 1, wherein the controller is configured to
control the light source to emit white light in the period other than the exposure period, and
control the light source to emit light having a wavelength band different from at least the white light in the exposure period.

4. The control device according to claim 3, wherein the light emitted by the light source in the exposure period is light of a wavelength range serving as part of a wavelength range of visible light.

5. The control device according to claim 3, wherein the light emitted by the light source in the exposure period is light in a wavelength range outside of a wavelength range of visible light.

6. The control device according to claim 1, further comprising an image processor configured to:
synthesize the image signals of the two temporally-continuous captured image frames to generate a display image for the display device;
detect a bright point position based on the image signal of one of the captured image frames; and
replace a pixel value of the detected bright point position with a pixel value of a position corresponding to the image signal of another captured image frame to synthesize the image signals of the two captured image frames.

7. The control device according to claim 6, wherein the controller is configured to:
control the light source to emit the illumination light in exposure processing of one of the captured image frames; and,
control the light source, in exposure processing of the other captured image frame, to emit the illumination light having a lower light intensity than the illumination light of the exposure processing of the one of the captured image frames.

8. The control device according to claim 7, wherein emission of the illumination light is stopped in the exposure processing of the other captured image frame.

9. The control device according to claim 1, wherein the image sensor has a global shutter function configured to read an electric charge of the light receiving element serving as a reading target at one time.

10. A medical observation system comprising:
an imaging device including an image sensor configured to capture an image of a subject;
a support configured to support the imaging device;
a light source configured to irradiate the subject with illumination light;
a control device configured to control the imaging device and the light source; and
a display configured to display the image captured by the imaging device, wherein
the control device is configured to change at least part of an illumination condition of the illumination light of an exposure period of a light receiving element of the image sensor and a period other than the exposure period in an image-capturing processing period for acquiring an image signal of a one frame image displayed by the display.
